(19) United States
(12) Patent Application Publication
(10) Pub. No.: US 2025/0224858 A1
(43) Pub. Date: Jul. 10, 2025

(54) SYSTEMS, METHODS, AND DEVICES FOR AUTOMATED CELLULAR THERAPY MANUFACTURING THROUGH GRAPHICAL USER INTERFACE

(71) Applicant: Multiply Labs Inc., San Francisco, CA (US)

(72) Inventors: Roberto SERRA, Torino (IT); Stefano DESSl, Cagliari (IT); Panagiotis KARAGIANNIS, Milano (IT); Giorgio MARITANO, Torino (IT); Danilo CIAFFI, Rome (IT); Maxwell RADEMACHER, Boston, MA (US)

(21) Appl. No.: 19/014,061

(22) Filed: Jan. 8, 2025

Related U.S. Application Data
(60) Provisional application No. 63/618,851, filed on Jan. 8, 2024.

(51) Int. Cl. G06F 3/04842; G05B 19/418; G06F 3/0481

(52) U.S. Cl. CPC G06F 3/04842; G05B 19/41865; G06F 3/0481

(57) ABSTRACT

Systems, methods, and devices for facilitating automated manufacture of one or more cellular therapies through a graphical user interface (GUI) are provided. A method includes displaying a plurality of objects, each associated with a unit operation to be performed by a robotic cluster of a biological foundry. The method also include selecting and organizing desired objects to generate a representation of a workflow for the robotic cluster(s) to produce some or all of a cellular therapy. The representation of a workflow can be saved, retrieved and/or modified to generate an updated representation of the workflow.

### CROSS-REFERENCE TO RELATED APPLICATION

The present Application claims priority to U.S. Provisional Patent Application No. 63/618,851, entitled “SYSTEMS, METHODS, AND DEVICES FOR AUTOMATED CELLULAR THERAPY MANUFACTURING THROUGH GRAPHICAL USER INTERFACE,” filed Jan. 8, 2024, which is hereby incorporated by reference in its entirety for all purposes.

### TECHNICAL FIELD

The present invention is directed to systems, methods, and devices for automated cellular therapy manufacture through a graphical user interface. More particularly, the present disclosure is directed to systems, methods, and devices for creating, generating, modifying, updating, and/or compiling a workflow for automated cellular therapy manufacture using a graphical user interface.

### BACKGROUND

Traditionally, cell therapies are produced with labor-intensive processes. These conventional processes require not only a large number of manufacturing operators, but also the employment of highly skilled (and expensive) technicians. These constraints make it particularly difficult to manufacture cell therapies at an industrial scale. For instance, a large number of conventional solutions for manufacturing cell therapies have been focused on optimization of subsystems and/or more precise, real-time data collection, and a greater level of automation and integration of advanced robotics technologies. See, Rüßmann et al., 2015, “Industry 4.0: The future of productivity and growth in manufacturing industries,” Boston Consulting Group, 9(1), 54.

Biological foundries are being increasingly used to automate and handle these applications. Biological foundries can be complex and expensive. Moreover, efficient use of such foundries presents a difficult scheduling problem. For instance, two different processes operating at the foundry may need to use the same instrument. Without some consideration for scheduling, conflicts may arise where two different processes request the same instrument. Moreover, without some consideration for scheduling, the foundry may be under-utilized, with the foundry proceeding to process tasks at some form of lowest common denominator associated with the foundry. Further, for handling sensitive ells, intricate processes and precision are needed, which conventionally requires manually handling, connecting, and double-checking complex closed-system and the like.

Given the above background, what is needed in the art are improved systems, methods, and devices for automated manufacture of cell therapies.

The information disclosed in this background section is only for enhancement of understanding of the general background of the invention and should not be taken as an acknowledgement or any form of suggestion that this information forms the prior art already known to a person skilled in the art.

### SUMMARY

The present disclosure addresses the shortcomings disclosed above and/or other problems by providing systems, methods, and devices that facilitate automated manufacture of one or more cellular therapies through unique user interfaces for electronic devices (or more generally, computer systems).

In some embodiments, the systems, methods, and devices of the present disclosure provide a graphical user interface that allows for the automated manufacture of one or more cell therapies. For instance, in some embodiments, the graphical user interface of the present disclosure allows for an end-user to easily program a workflow to manufacture a first cell therapy, such as by modifying a workflow to generate an updated workflow in order to change how one or more instruments manufacture the first cell therapy. In some embodiments, the graphical user interface enables the end-user to design the workflow to manufacture the first cell therapy from a plurality of basic building blocks, which are visually represented by a plurality of objects that correspond to the plurality of basic building blocks. Collectively, the plurality of objects specifies operations for the one or more instruments, such as one or more articulated handling robots, to perform in order to manufacture the first cell therapy. In this way, the systems and methods of the present disclosure allow for manufacture of the first cell therapy in an automated and modular fashion that does not require end users to make substantial modifications to original manufacturing processes when performed by hand or the like. In this way, the systems and methods of the present disclosure mitigate the risk of human error while simultaneously boosting cell therapy output. The systems and methods of the present disclosure allow for executing workflows that is visualized to a user via a display, which can be run without human intervention, allowing manufacturing operators and process scientists to oversee the production of multiple therapies at once, without being tied down in the delicate, tedious, and mission-critical manual tasks involved in the traditional manufacturing process, driving significant benefits around efficiency, and ultimately support the scalability of cell therapies.

In some embodiments, the systems, methods, and devices of the present disclosure provide an optimization algorithm. In some embodiments, the optimization algorithm of the present disclosure scales the workflow to allow the robotic cluster to efficiently manufacture multiple cellular therapies in parallel.

In various embodiments, the present disclosure is directed to providing a method for facilitating automated manufacture of one or more cellular therapies through a graphical user interface (GUI). The method includes A) displaying, through the GUI of a display, a plurality of objects in a first region of the GUI. Each object in the plurality of objects is associated with a unit operation to be performed by a robotic cluster of a biological foundry. The method also includes B) generating a representation of a workflow for producing some or all of a first cellular therapy in the one or more cellular therapies. In some embodiments, the generating includes B.1) selecting, through the GUI of the display, a desired object from the plurality of objects displayed in the first region of the GUI, B.2) placing, through the GUI of the display, the desired objects in a second region of the GUI, and B.3) repeating the selecting B.1) and the placing B.2) until each object in a first set of objects is selected and placed in the second region of the GUI. The first set of objects is organized in an order, thereby generating the representation of the workflow for producing some or all of the first cellular therapy.

In some embodiments, the plurality of objects includes a first object that is associated with a first instruction to start the workflow, a second object that is associated with a second instruction to end the workflow, or both of the first object and the second object.

In some such embodiments, the first set of objects includes the first object, the second object, or both of the first object and the second object.

In some embodiments, the first set of objects includes two or more objects selected from the plurality of objects.

In an exemplary embodiment, the first set of objects consists of each object in the plurality of objects.

In some embodiments, the plurality of objects includes one or more enrich objects, one or more expand objects, one or more transfer objects, one or more harvest objects, one or more freeze objects, one or more formulate objects, one or more add reagent objects, one or more concentration-based sample objects, one or more fixed volume sample objects, or any combination thereof.

In some embodiments, the selecting B.1) and the placing B.2) are conducted by clicking the desired object in the plurality of objects displayed in the first region of the GUI and dragging the desired object to the second region of the GUI.

In some embodiments, the generating further includes: B.4) setting or editing, subsequent to the selecting, the placing or the repeating, one or more parameters for a respective object in the first set of objects.

In some embodiments, a respective object in the plurality of objects includes a plurality of corresponding sub-objects.

In some such embodiments, the method further includes: C) displaying, through the GUI of the display, the plurality of corresponding sub-objects of the respective object in a third region of the GUI.

In some embodiments, the present disclosure is directed to providing a method for facilitating automated manufacture of one or more cellular therapies through a graphical user interface. In some embodiments, the method includes displaying, through the graphical user interface of a display, a representation of a workflow. The representation of the workflow is configured to produce some or all of a first cellular therapy in the one or more cellular therapies. Moreover, the representation includes a plurality of objects displayed in a first sequence. In some embodiments, the method further includes receiving, through the graphical user interface of the display, a modification to the representation of the workflow. Additionally, in some embodiments, the method includes further displaying, through the graphical user interface of the display, an updated representation of the workflow. In some embodiments, the updated representation includes some or all of the plurality of objects displayed in a second sequence different from the first sequence.

In some embodiments, the representation of the workflow defines an order of operations for a remote device to perform.

In some embodiments, the order of operations includes a sort operation, an edit operation, a culture operation, a quality control operation, a formulate operation, or a combination thereof.

In some embodiments, the order of operations is spatiotemporally organized.

In some embodiments, the workflow is configured to be performed concurrently with a different workflow.

In some embodiments, the plurality of objects includes one or more tiles, one or more squares, one or more rectangles, one or more closed-form polygons, one or more circles, or more ellipses, or a combination thereof.

In some embodiments, each object in the plurality of objects is associated with a unit operation performed by the remote device.

In some embodiments, the receiving the modification further includes determining if the modification satisfies one or more heuristic constraints.

In some embodiments, the modification includes a reagent volume associated with a corresponding object in the plurality of objects.

In some embodiments, the modification includes a location associated with a corresponding object in the plurality of objects.

In some embodiments, the present disclosure is directed to providing a computer system. The computer system includes a display, one or more processors, and memory. The memory stores instructions for executing a method of the present disclosure.

In some embodiments, the present disclosure is directed to providing a computer readable storage medium. The computer readable storage medium stores one or more programs.

Moreover, the one or more programs includes instructions, which when executed by an electronic device with one or more processors and a memory cause the electronic device to execute a method of the present disclosure.

### BRIEF DESCRIPTION OF THE DRAWINGS

FIG. 1 illustrates various modules and/or components of a computer system, in accordance with an embodiment of the present disclosure;

FIG. 2 depicts a view of a graphical user interface for facilitating automated manufacture of one or more cellular therapies, in accordance with an embodiment of the present disclosure;

FIG. 3 depicts another view of a graphical user interface for facilitating automated manufacture of one or more cellular therapies, in accordance with an embodiment of the present disclosure;

FIG. 4 depicts yet another view of a graphical user interface for facilitating automated manufacture of one or more cellular therapies, in accordance with an embodiment of the present disclosure;

FIG. 5 depicts yet another view of a graphical user interface for facilitating automated manufacture of one or more cellular therapies, in accordance with an embodiment of the present disclosure;

FIGS. 6A and 6B collectively provide a flow chart of methods for facilitating automated manufacture of one or more cellular therapies through a graphical user interface, in which optional steps or embodiments are indicated by dashed boxes;

FIG. 7 illustrates a schematic view of a biological foundry for manufacturing one or more cellular therapies using one or more instruments including an articulated handling robot, in accordance with an embodiment of the present disclosure;

FIG. 8 illustrates various logic functions that are implemented by a computer system in some embodiments of the present disclosure; and

FIG. 9 provide a flow chart of methods for facilitating automated manufacture of one or more cellular therapies through a graphical user interface, in which optional steps or embodiments are indicated by dashed boxes.

It should be understood that the appended drawings are not necessarily to scale, presenting a somewhat simplified representation of various features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION

Systems, methods, and devices for facilitating automated manufacture of one or more cellular therapies through a graphical user interface (GUI) are provided. For instance, in some embodiments, the systems, methods, and devices of the present disclosure allow for creating or modifying one or more workflows at a biological foundry by applying various abstractions of architectures and designs of manufacturing systems to plan a sequence of manufacturing actions in the form of a representation of a workflow describes successful manufacture of some or all of a cellular therapy.

In some embodiments, a plurality of objects is displayed, with each object is associated with a unit operation to be performed by a robotic cluster of a biological foundry. Desired objects can be selected, edited and/or organized to generate a representation of a workflow, thereby allowing a user to easily “program” the behavior of the robotic cluster(s) to produce their own cell therapy product.

In some embodiments, the representation of the workflow is displayed through the GUI of a display. The representation includes a plurality of objects displayed in a first sequence, which provides an order to the workflow. A modification to the representation of the workflow is received through the GUI. From this, an updated representation of the workflow is further displayed through the GUI. The updated representation includes some or all of the plurality of objects displayed in a second sequence different from the first sequence, which represents a change in the order of operations of the workflow.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, and components have not been described in detail so as not to unnecessarily obscure aspects of the embodiments.

It will also be understood that, although the terms first, second, etc. may be used herein to describe various elements, these elements should not be limited by these terms. These terms are only used to distinguish one element from another. For instance, a first property could be termed a second property, and, similarly, a second property could be termed a first property, without departing from the scope of the present disclosure. The first property and the second property are both properties, but they are not the same property.

The terminology used in the present disclosure is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The foregoing description included example systems, methods, techniques, instruction sequences, and computing machine program products that embody illustrative implementations. For purposes of explanation, numerous specific details are set forth in order to provide an understanding of various implementations of the inventive subject matter. It will be evident, however, to those skilled in the art that implementations of the inventive subject matter may be practiced without these specific details. In general, well-known instruction instances, protocols, structures, and techniques have not been shown in detail.

The foregoing description, for purpose of explanation, has been described with reference to specific implementations. However, the illustrative discussions below are not intended to be exhaustive or to limit the implementations to the precise forms disclosed. Many modifications and variations are possible in view of the above teachings. The implementations are chosen and described in order to best explain the principles and their practical applications, to thereby enable others skilled in the art to best utilize the implementations and various implementations with various modifications as are suited to the particular use contemplated.

In the interest of clarity, not all of the routine features of the implementations described herein are shown and described. It will be appreciated that, in the development of any such actual implementation, numerous implementation-specific decisions are made in order to achieve the designer's specific goals, such as compliance with use case-and business-related constraints, and that these specific goals will vary from one implementation to another and from one designer to another. Moreover, it will be appreciated that such a design effort might be complex and time-consuming, but nevertheless be a routine undertaking of engineering for those of ordering skill in the art having the benefit of the present disclosure.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. Where particular values are described in the application and claims, unless otherwise stated, the term “about” means within an acceptable error range for the particular value. The term “about” can have the meaning as commonly understood by one of ordinary skill in the art. The term “about” can refer to ±10%. The term “about” can refer to ±5%.

Furthermore, when a reference number is given an “i^{th}” denotation, the reference number refers to a generic component, set, or embodiment. For instance, an instrument termed “instrument i” refers to the i^{th} instrument in a plurality of instrument (e.g., an instrument 52-i in a plurality of instruments 52).

In some embodiments, the processes described below enhance the operability of the devices and make the user-device interfaces more efficient (e.g., by helping the user to provide proper inputs and reducing user mistakes when operating and/or interacting with the device) through various techniques, including by providing improved visual feedback to the user, reducing the number of inputs needed to perform an operation, providing additional control options without cluttering the user interface with additional displayed controls, performing an operation when a set of conditions has been met without requiring further user input, and/or additional techniques. In some embodiments, these techniques also reduce power usage and improve battery life of the device by enabling the user to use the device more quickly and efficiently.

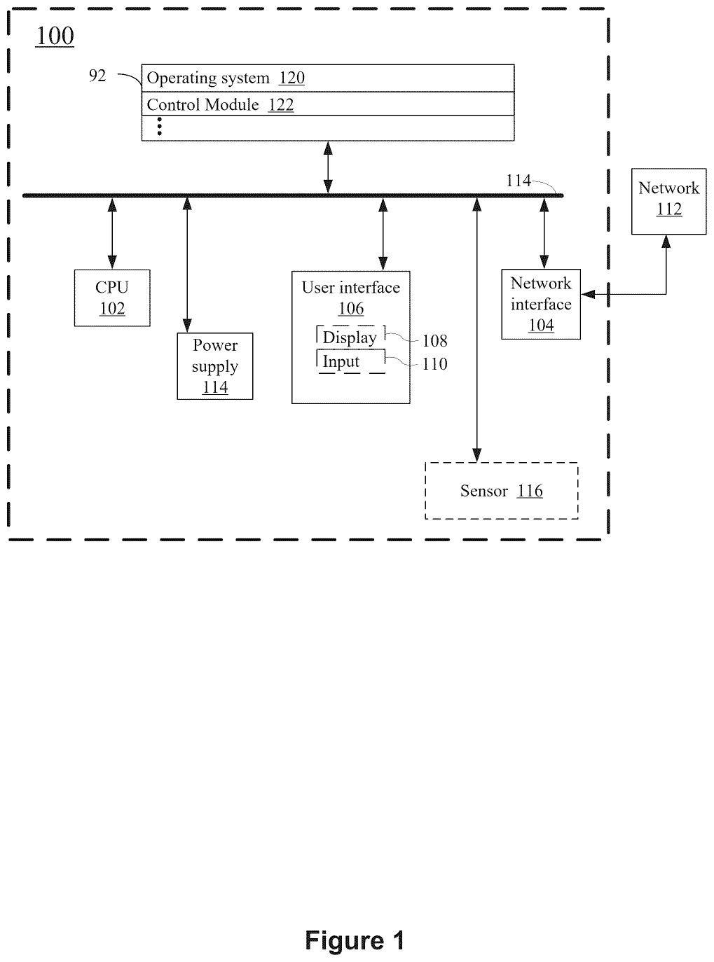

In the present disclosure, unless expressly stated otherwise, descriptions of devices and systems will include implementations of one or more computers. For instance, and for purposes of illustration in FIG. 1, a computer system 100 is represented as single device that includes all the functionality of the computer system 100. However, the present disclosure is not limited thereto. For instance, in some embodiments, the functionality of the computer system 100 is spread across any number of networked computers and/or reside on each of several networked computers and/or by hosted on one or more virtual machines and/or containers at a remote location accessible across a communications network (e.g., communications network 112 of FIG. 1). One of skill in the art will appreciate that a wide array of different computer topologies is possible for the computer system 100, and other devices and systems of the preset disclosure, and that all such topologies are within the scope of the present disclosure. Moreover, rather than relying on a physical communication network 112, the illustrated devices and systems may wirelessly transmit information between each other. As such, the exemplary topology shown in FIG. 1 merely serves to describe the features of an embodiment of the present disclosure in a manner that will be readily understood to one of skill in the art.

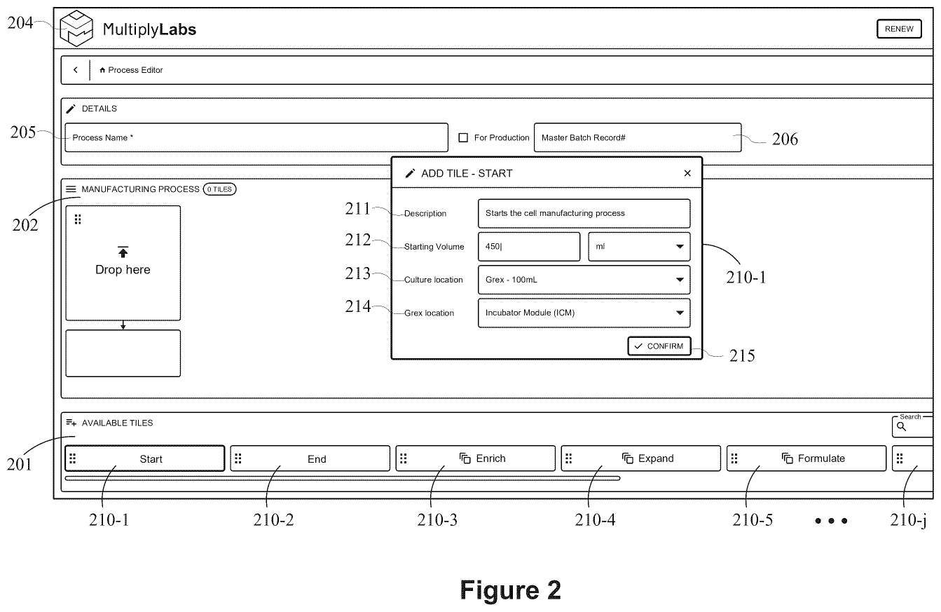

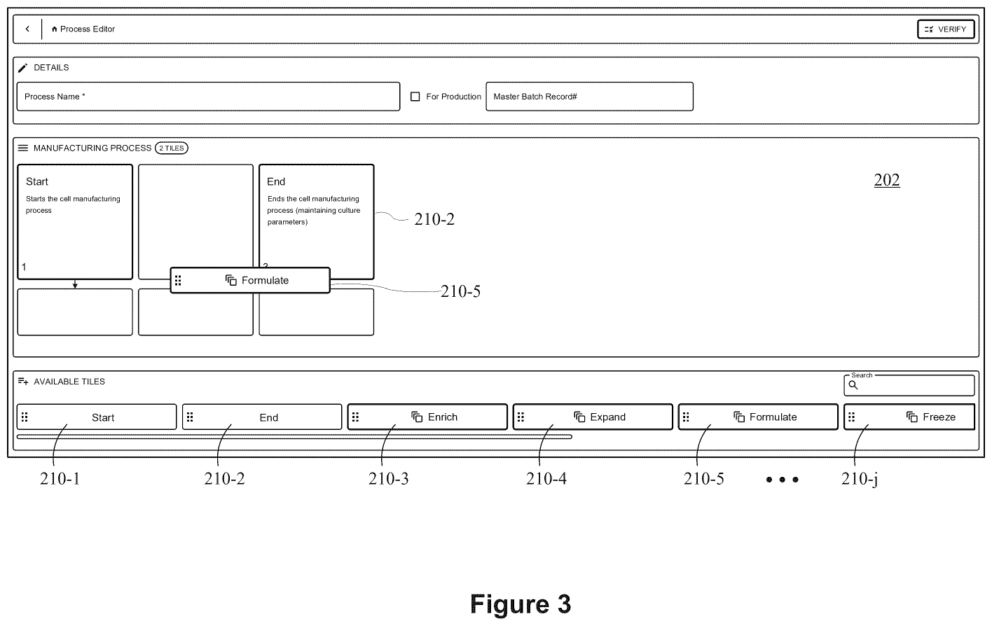

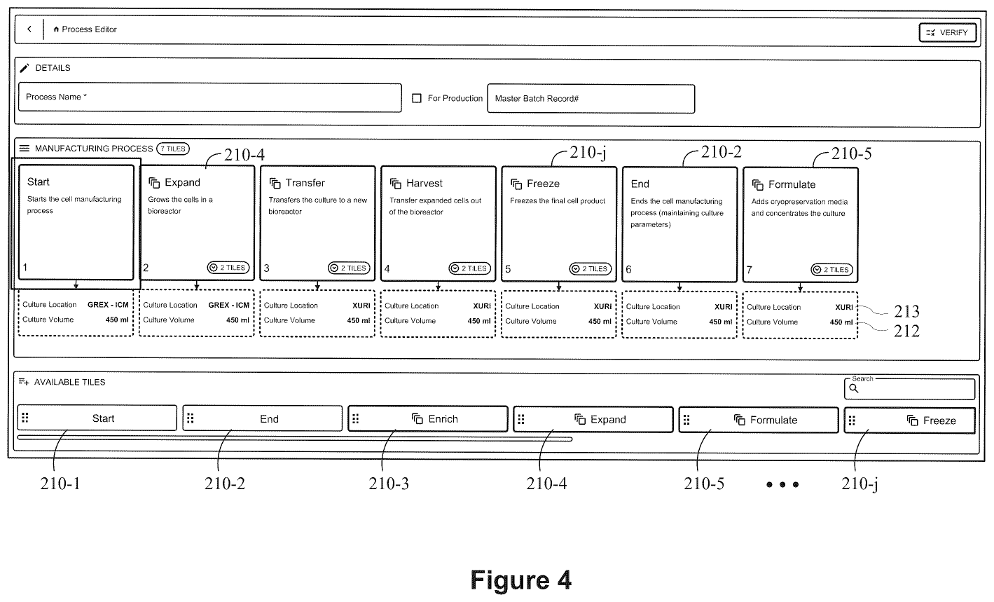

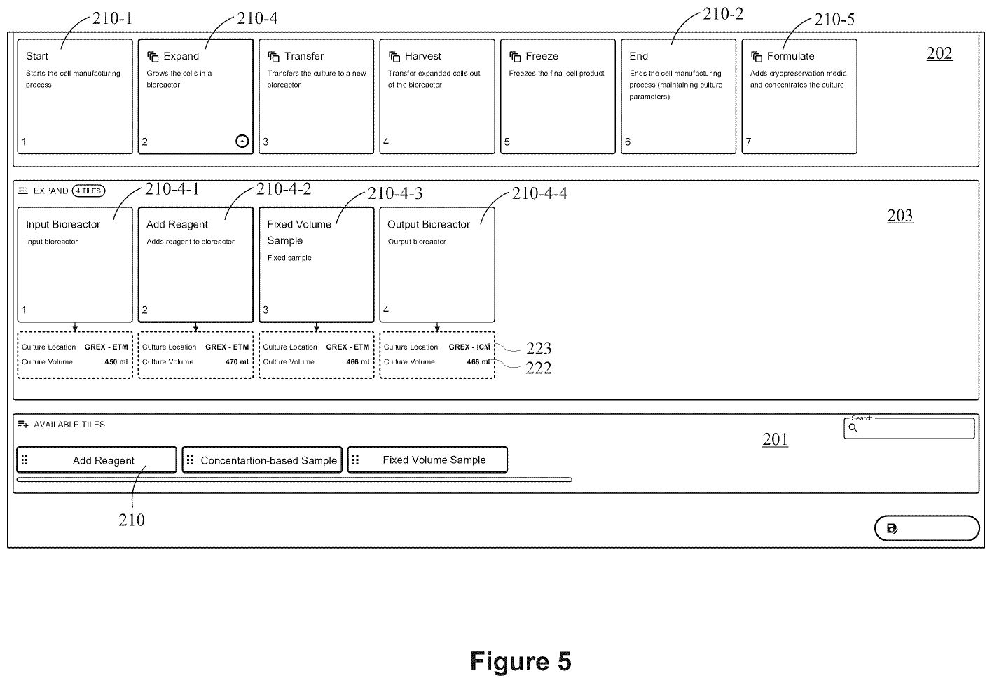

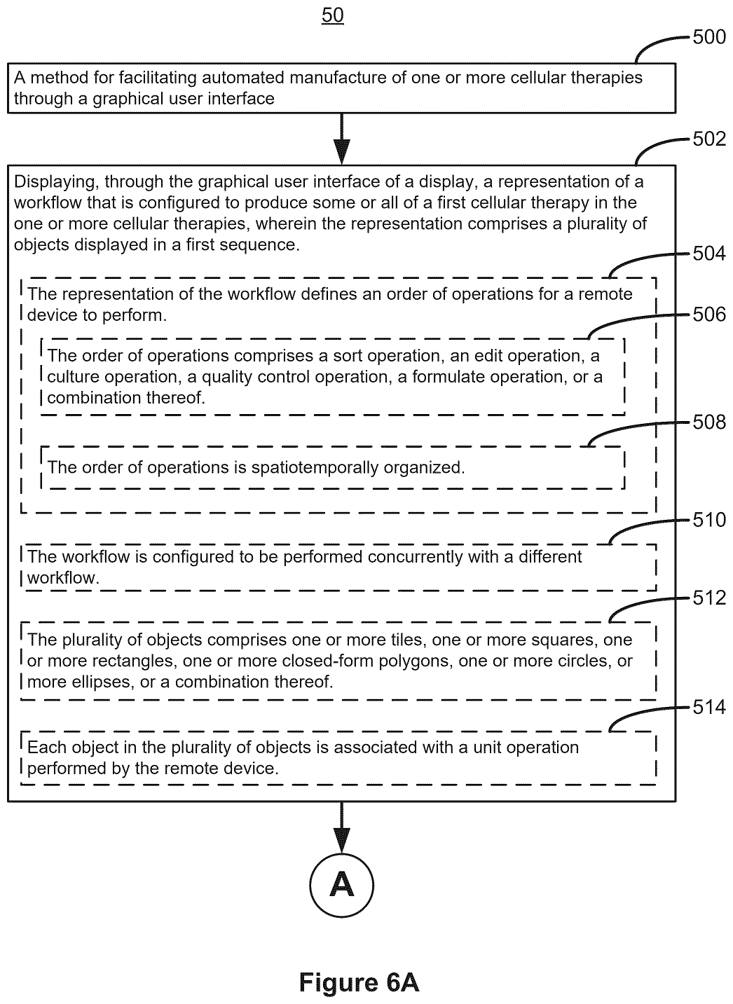

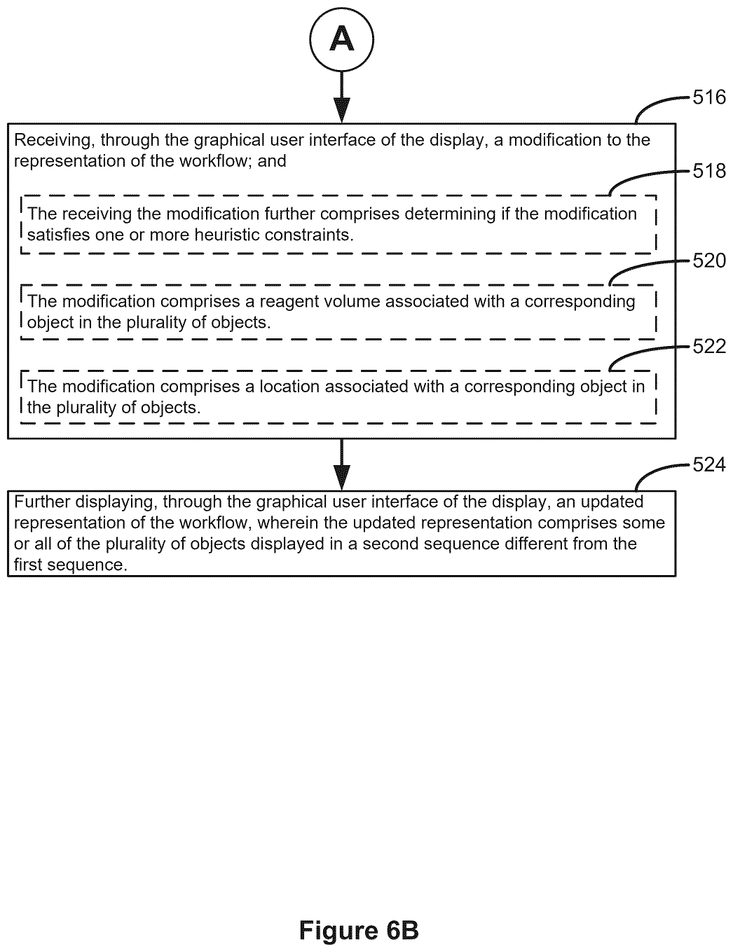

FIG. 1 depicts a block diagram of a distributed computer system (e.g., computer system 100) according to some embodiments of the present disclosure. The computer system 100 at least facilitates communicating one or more instructions for facilitating automated manufacture of one or more cellular therapies through a graphical user interface (e.g., graphical user interface of FIG. 2, graphical user interface of FIG. 3, graphical user interface of FIG. 4, graphical user interface of FIG. 5, graphical user interface of method 50 of FIG. 6, etc.).

In some embodiments, the communication network 112 optionally includes the Internet, one or more local area networks (LANs), one or more wide area networks (WANs), other types of networks, or a combination of such networks.

Examples of communication networks 112 include the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

In various embodiments, the computer system 100 includes one or more processing units (CPUs) 102, a network or other communications interface 104, and memory 92.

In some embodiments, the computer system 100 includes a user interface 106. The user interface 106 typically includes a display 108 for presenting media. In some embodiments, the display 108 is integrated within the computer systems (e.g., housed in the same chassis as the CPU 102 and memory 112). In some embodiments, the computer system 100 includes one or more input device(s) 110, which allow a subject to interact with the computer system 100. In some embodiments, input devices 110 include a keyboard, a mouse, and/or other input mechanisms. Alternatively, or in addition, in some embodiments, the display 108 includes a touch-sensitive surface (e.g., where display 108 is a touch-sensitive display or computer system 100 includes a touch pad).

In some embodiments, the computer system 100 presents media to a user through the display 108. Examples of media presented by the display 108 include one or more images, a video, audio (e.g., waveforms of an audio sample), or a combination thereof. In typical embodiments, the one or more images, the video, the audio, or the combination thereof is presented by the display 108 through a client application (e.g., client application 124 of FIG. 1). In some embodiments, the audio is presented through an external device (e.g., speakers, headphones, input/output (I/O) subsystem, etc.) that receives audio information from the computer system 100 and presents audio data based on this audio information. In some embodiments, the user interface 106 also includes an audio output device, such as speakers or an audio output for connecting with speakers, earphones, or headphones.

Memory 92 includes high-speed random access memory, such as DRAM, SRAM, DDR RAM, or other random access solid state memory devices, and optionally also includes non-volatile memory, such as one or more magnetic disk storage devices, optical disk storage devices, flash memory devices, or other non-volatile solid state storage devices. Memory 92 may optionally include one or more storage devices remotely located from the CPU(s) 102. Memory 92, or alternatively the non-volatile memory device(s) within memory 92, includes a non-transitory computer readable storage medium. Access to memory 92 by other components of the computer system 100, such as the CPU(s) 102, is, optionally, controlled by a controller. In some embodiments, memory 92 can include mass storage that is remotely located with respect to the CPU(s) 102. In other words, some data stored in memory 92 may in fact be hosted on devices that are external to the computer system 100, but that can be electronically accessed by the computer system 100 over an Internet, intranet, or other form of communication network 112 or electronic cable using communication interface 104.

In some embodiments, the memory 92 of the computer system 100 stores:
an operating system 120 (e.g., ANDROID, iOS, DARWIN, RTXC, LINUX, UNIX, OS X, WINDOWS, or an embedded operating system such as VxWorks) that includes procedures for handling various basic system services;
an optional electronic address associated with the computer system 100 that identifies the computer system 100 (e.g., within the communication network 112);
a control module 122 for controlling one or more instruments (e.g., a metering device, a gripper device, a nest device, a calibration device, a docking device, a mounting device, a tray device, a tablet deposition device, or a combination thereof) associated with the computer system 100; and
a client application for presenting information (e.g., media) using a display 108 of the computer system 100.

As indicated above, in some embodiments, an electronic address is associated with the computer system 100. The electronic address is utilized to at least uniquely identify the computer system 100 from other devices and components of the distributed system 100, such as other devices having access to the communications network 112.

In some embodiments, a control module 122 is utilized for controlling one or more controlling one or more instruments 52 (e.g., a metering device, a gripper device, a nest device, a calibration device, a docking device, a mounting device, a tray device, a tablet deposition device, or a combination thereof) associated with the computer system 100 and/or for controlling performance of one or more workflows performed, at least in part, at or using the one or more instruments 52. For instance, in some embodiments, the one or more instruments 52 includes an articulated handling robot that is configured to advance the one or more workflows between a first instrument 52-1 and a second instrument 52-2 in the one or more instruments 52 at a biological foundry. However, one of skill in the art will appreciate that the present disclosure is not so limited.

In some embodiments, the computer system 100 optionally also includes one or more sensors 116, such as one or more optical sensors 116 to allow for visual inspection of a respective instrument. The optical sensor(s) 116 optionally include charge-coupled device (CCD) or complementary metal-oxide semiconductor (CMOS) phototransistors. The optical sensor(s) 116 optionally capture still images and or video, such as an image of a first cellular therapy and/or a volume of a first container associated with the computer system.

In some embodiments, the systems, methods, and apparatuses of the present disclosure include one or more systems, methods, apparatuses, and devices of United States Patent Publication no.: 2023-0102750 A1, entitled “SYSTEMS AND METHODS FOR FACILITATING MODULAR AND PARALLELIZED MANUFACTURING AT A BIOLOGICAL FOUNDRY,” filed Aug. 17, 2022; United States Patent Publication no.: 2022-0325219 A1, entitled “SYSTEM, METHOD, AND APPARATUS FACILITATING AUTOMATED MODULAR MANUFACTURE OF CELL THERAPY,” filed Dec. 13, 2021; United States Patent Publication no.: 2019-0016048 A1, entitled “SYSTEMS AND METHODS FOR DESIGNING AND MANUFACTURING MULTI-COMPARTMENT CAPSULES,” filed Jun. 18, 2018; U.S. Pat. No. 11,198,845, entitled “SYSTEM, METHOD, AND APPARATUS FACILITATING AUTOMATED MODULAR MANUFACTURE OF CELL THERAPY,” filed Apr. 19, 2021; U.S. Pat. No. 10,773,392, entitled “FLEXURE GRIPPING DEVICE,” filed Mar. 7, 2019; U.S. Pat. Nos. 11,142,353; 9,845,167, entitled “AUTOMATED BATCH FILLING APPARATUS,” filed Mar. 7, 2019; U.S. Pat. No. 10,456,975, entitled “MULTI-COMPARTMENT CAPSULE,” filed Jun. 16, 2017; U.S. patent application Ser. No. 18/306,936, entitled “SYSTEM, METHOD, AND APPARATUS FOR MANUFACTURE OF ENGINEERED CELLS,” filed Apr. 25, 2023; U.S. patent application Ser. No. 18/154,950, entitled “SYSTEM, METHOD, AND APPARATUS FOR MANUFACTURE OF ENGINEERED CELLS,” filed Jan. 16, 2023; and U.S. patent application Ser. No. 17/823,065, entitled “SYSTEM, METHOD, AND APPARATUS FACILITATING ASSEMBLY OF A CAPSULE,” filed Aug. 29, 2022, each of which is hereby incorporated by reference in its entirety for all purposes.

Now that details of a computer system 100 for facilitating automated manufacture of one or more cellular therapies through a graphical user interface have been disclosed, details regarding a flow chart of processes and features for implementing a method 50, in accordance with an embodiment of the present disclosure, are disclosed with reference to FIGS. 6A and 6B.

Referring to block 500 of FIG. 6A, in some embodiments, a method 50 for facilitating automated manufacture of one or more cellular therapies through a graphical user interface (e.g., graphical user interface of FIG. 2, graphical user interface of FIG. 3, graphical user interface of FIG. 4, graphical user interface of FIG. 5, graphical user interface of method 50 of FIGS. 6A-6B, etc.) is provided.

In some embodiments, a cellular therapy in the one or more cellular therapies is an assembly of nucleic acid components. For instance, in some embodiments, the cellular therapy is a plasmid, and the nucleic acid components are predetermined promoters, repressors, stop codon, and exons. In some embodiments, the cellular therapy is a different predetermined nucleic acid with a different predetermined nucleic acid sequence. In some embodiments, the cellular therapy is a different predetermined ribonucleic acid (mRNA) with a different predetermined nucleic acid sequence. In some embodiments, the cellular therapy is a different predetermined deoxyribonucleic acid (DNA) with a different predetermined nucleic acid sequence. In some embodiments, the cellular therapy is a different predetermined polymer. In some embodiments, the cellular therapy is a different predetermined peptide. In some embodiments, the cellular therapy is a different predetermined protein.

As such, in the context of biological foundries, the cellular therapy is one of the objectives of a research and development project that defines the desired biological trait to be achieved by a workflow. The cellular therapy can be either quantitative or qualitative. For example, in one embodiment, the cellular therapy can be a genetic configuration for a biosynthetic pathway that produces more compound of interest than a current level. In another embodiment, the cellular therapy is a genetic configuration for a microbial host that has a tolerance to an inhibitor over X mg/L. Additionally, in some embodiments the cellular therapy is a polynucleotide or nucleic acid sequence.

Referring to block 502, in some embodiments, the method 50 includes displaying a representation of a workflow through a display (e.g., display 108 of FIG. 1). In some embodiments, the workflow is configured to produce some or all of a first cellular therapy in the one or more cellular therapies, such that the representation of the workflow provides a visualization of the process to manufacture some or all of the first cellular therapy.

In some embodiments, each workflow is a manufacturing process that produces one or more cellular therapies. In some embodiments, to form a representation of a workflow, the systems and methods of the present disclosure analyzes the manufacturing process by breaking the manufacturing process into a corresponding plurality of operations. For each operation, there is a corresponding instrument 52, or action, which could be a realization of many possible embodiments of the corresponding instrument 52. Each instrument 52 is controlled by a standardized control module attached to the respective instrument 52. This control module receives a plurality of commands and transmits data about a status of executing an instant of a corresponding workflow conducted by the instrument 52. In some embodiments, a respective instrument 52 will perform different operations depending on the commands provided to the respective instrument 52 and the cellular therapy for that workflow. For example, in some embodiments, an aseptic bottle filling instruments 52-1 fills a specific compound of a respective formulation, and depending on a correspond position in a temporal order of the plurality of operations based on the cellular therapy, that plurality of operations to produce the formulation of the cellular therapy will call for various volumes of liquid. Consider, a bottle A instrument 52-2 is input to the filling instrument 52-1, and filled will 100 milliliters (mL) and then removed and processed by a first weigh-checker instrument 52-3, once the bottle A 52-2 is removed from the bottle filling instrument 52-1, a bottle B 52-4 is input into the bottle filling instrument 52-1 and filled with 250 mL and then processed by a second weigh-checker instrument 52-5. However, the present disclosure is not so limited, as this concept translates across a plurality of workflow operations and corresponding operations including powder filling, tableting, machining depths, pick-and-place operations, etc. According, the systems and method of the present disclosure allow for customization of the instruments 52 used for executing the representation of the workflow, being designed with the capacity to flexibly adapt to changes in temporal order and/or process, or spatial order.

Moreover, in some embodiments, the representation of the workflow includes a plurality of objects. In some embodiments, the plurality of objects is displayed through the graphical user interface in a first sequence. For instance, referring briefly to FIG. 5, in some embodiments, the plurality of objects includes one or more square objects that is arranged in a linear order from a first block to a seventh block. However, the present disclosure is not limited thereto.

In some embodiments, the plurality of objects consists of between 2 and 20 objects, 2 and 11 objects, 3 and 19 objects, 3 and 10 objects, 4 and 18 objects, 4 and 9 objects, 5 and 17 objects, 5 and 8 objects, 6 and 16 objects, 6 and 7 objects, 7 and 15 objects, 8 and 14 objects, 9 and 13 objects, 10 and 12 objects, 11 and 20 objects, 12 and 19 objects, 13 and 18 objects, 14 and 17 objects, or 15 and 16 objects.

In some embodiments, the plurality of objects includes at least 2 objects, at least 3 objects, at least 4 objects, at least 5 objects, at least 6 objects, at least 7 objects, at least 8 objects, at least 9 objects, at least 10 objects, at least 11 objects, at least 12 objects, at least 13 objects, at least 14 objects, at least 15 objects, at least 16 objects, at least 17 objects, at least 18 objects, at least 19 objects, or at least 20 objects.

In some embodiments, the plurality of objects includes 2 objects, at most 3 objects, at most 4 objects, at most 5 objects, at most 6 objects, at most 7 objects, at most 8 objects, at most 9 objects, at most 10 objects, at most 11 objects, at most 12 objects, at most 13 objects, at most 14 objects, at most 15 objects, at most 16 objects, at most 17 objects, at most 18 objects, at most 19 objects, or at most 20 objects.

Referring to block 504, in some embodiments, the representation of the workflow defines an order of operations for a remote device to perform. For instance, in some embodiments, the order of operations is arranged such that a first operation is configured as a first terminus operation and/or a second operation is configured as a second terminus operation in the order of operations. In some embodiments, the order of operations provides a temporal and/or spatial order for performing one or more tasks associated with the workflow. However, the present disclosure is not limited thereto.

In some embodiments, the order of operations provides a charting of each step or process that collectively produces the first cellular therapy. In some embodiments, the charting allows for a subject to visualize some or all of the order of operations, such as when configuring and/or executing the representation of the workflow.

Referring to block 506, in some embodiments, the order of operations includes a sort operation, an edit operation, a culture operation, a quality control operation, a formulate operation, or a combination thereof.

Referring to block 508, in some embodiments, the order of operations is spatiotemporally organized. From this spatiotemporally ordering, each step or process in the workflow that is required to complete manufacture of the first cellular therapy is associated with a temporal order and a spatial position, such as within a biological foundry. For instance, in some embodiments, the order of operations allows for the computer system to perform the workflow in which a “fast” modules instruments feed multiple parallel bioreactor modules instruments. However, the present disclosure is not limited thereto.

Referring to block 510, in some embodiments, the workflow is configured to be performed concurrently with a different workflow. For instance, in some embodiments, the workflow is performed as part of a series workflow, such that a portion of the temporal order of the workflow cannot be modified. In some embodiments, the workflow is performed as part of a parallel workflow. However, the present disclosure is not limited thereto.

Referring to block 512, in some embodiments, the plurality of objects includes one or more tiles, one or more squares, one or more rectangles, one or more closed-form polygons, one or more circles, or more ellipses, or a combination thereof. In this way, in some embodiments, each respective object in the plurality of objects is visualized using a closed form shape, such that the respective object includes an edge or boundary having a similar or substantially similar shape as that of an adjacent object in the plurality of objects. However, the present disclosure is not limited thereto.

For instance, referring briefly to FIG. 2, in some embodiments, the plurality of objects includes a plurality of squares. Accordingly, in some embodiments, the representation of the workflow is depicted as a series of square tiles. In some embodiments, each respective square tile represents a corresponding basic building block (e.g., unit operation) to manufacture a respective cellular therapy and a series of actions the articulated handling robot will perform when executing the workflow. In some embodiments, each respective square tile represents a corresponding basic building block (e.g., unit operation) to manufacture a respective cellular therapy or a portion thereof and a series of actions the articulated handling robot will perform when executing the workflow

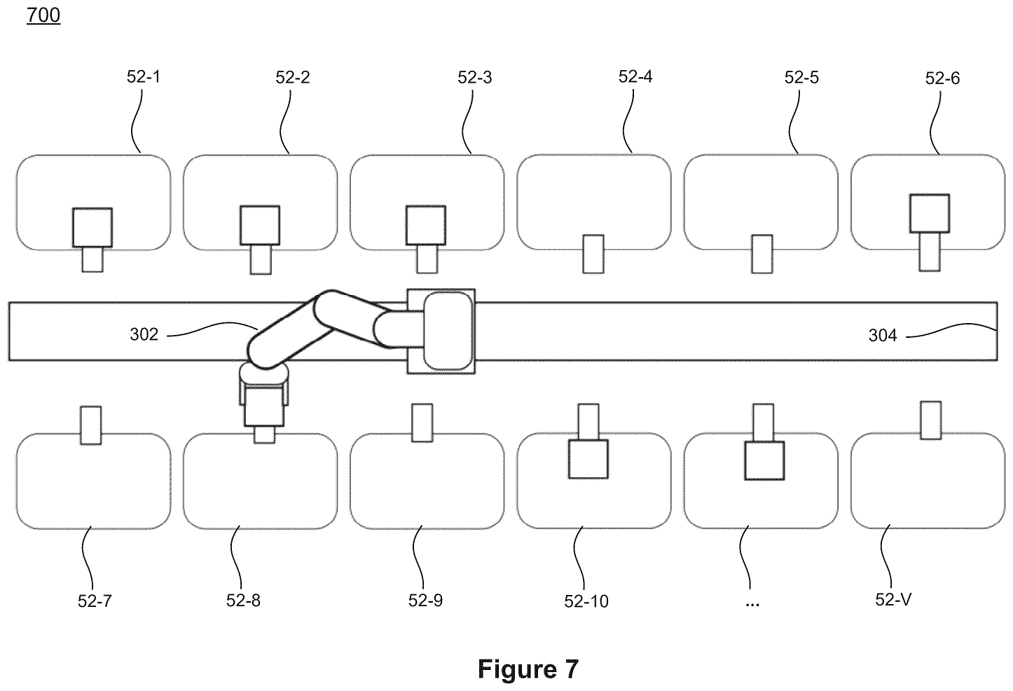

Referring to block 514, in some embodiments, each object in the plurality of objects is associated with a unit operation performed by the remote device, such as an articulated handling robot (e.g., articulated handling robot 302 of FIG. 7). Furthermore, in some embodiments, an object includes one or more subprocesses made up of objects in the plurality of objects, which allow for organization of complex processes through hierarchies of objects. For instance, referring briefly to FIG. 5, an “Expand” object in the plurality of objects includes one or more subprocesses of an “Input Bioreactor” object, an “Add Reagent” object, a “Fixed Volume Sample,” object, and an “Output Bioreactor” object. In some embodiments, a respective object is associated with a cell isolation task, such as by using an immunomagnetic bead-based instrument 52 for cell therapy manufacturing, which is a complex and time-consuming task to complete manually.

By incorporating this technology, cell therapy manufacturers can automate this part of the process without making substantial modifications to their original manufacturing processes. Automation in this deeply time-consuming area of the process will drive significant benefits around efficiency and support the scalability of cell therapies.

Referring to block 516 of FIG. 6B, in some embodiments, the method 50 includes receiving a modification to the representation of the workflow. In some embodiments, the modification to the representation is received through one or more inputs 110 from the computer system 100, such as one or more click and/or drag inputs 110 provided by an end-user of the computer system 100. For instance, in some embodiments, each object in the plurality of objects is editable, which allows for affecting the operations the one or more instruments 52 and/or the articulated handling robot 302 will perform when manufacturing the first cell therapy.

For instance, in some embodiments, the modification to the representation of the workflow is required through a plurality of inputs to generate the representation of the workflow. In some embodiments, the plurality of inputs includes a sequence of manufacturing steps for each process or step that produces the first cellular therapy. In some embodiments, the plurality of inputs includes a configuration of one or more instruments 52 associated with the biological foundry 700, such as a description of each instrument 52 associated with the biological foundry 700 that is further associated with at least one operation that manufactures the cell therapy. In some embodiments, the modification further includes a status of a respective instrument 52 in the one or more instruments 52, such as an estimated time to competition for an execution of an instance or a portion of the workflow. In some embodiments, the inputs include a total number of cellular therapies and/or a number of unique cellular therapies in the one or more cellular therapies manufactured by the workflow. From this, the representation of the workflow is provided with a temporal order sequence of manufacturing actions (e.g., transfers via articulated handling robot 302, actions at an instrument 52, etc.) describing successful manufacture of the cellular therapies.

For instance, referring briefly to FIG. 3, in some embodiments, the modification to the representation of the workflow is received by adding one or more objects from a bin of objects presented through the graphical user interface, such as receiving one or more click and drag inputs (e.g., through input 110 of FIG. 1).

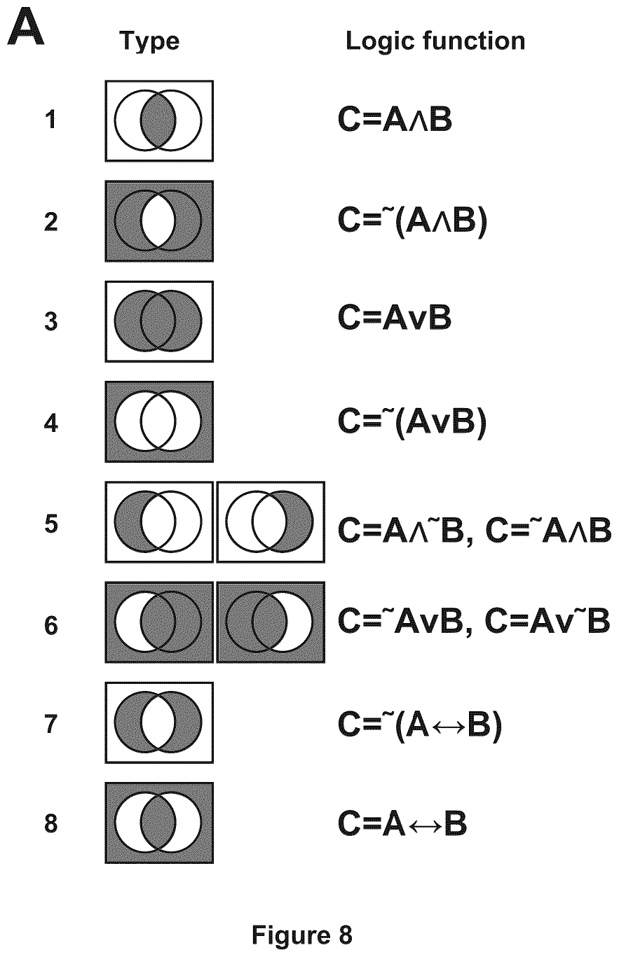

Referring to block 518, in some embodiments, the receiving the modification further includes determining if the modification satisfies one or more heuristic constraints. For instance, referring briefly to FIG. 8, in some embodiments, each heuristic constrain in the one or more heuristic constraints is implemented as one or more logic functions. As a non-limiting example, in some embodiments, a respective heuristic constraint is associated with a capacity of a corresponding instrument 52 (e.g., a maximum volume associated with a solution accommodated by the corresponding instrument 52, a maximum temperature associated with operating the corresponding instrument 52, a desensitization requirement prior to utilizing the corresponding instrument, etc.). However, the present disclosure is not limited thereto.

Referring to block 520, in some embodiments, the modification includes a reagent volume associated with a corresponding object in the plurality of objects. For instance, referring to FIG. 5, in some embodiments, the graphical user interface allows for tracking a volume of one or more containers as reagents are added and/or removed from the container, which is useful to understand fluid concentrations, such as in order to avoid overgrowth when manufacturing the first cell therapy.

Referring to block 522, in some embodiments, the modification includes a location associated with a corresponding object in the plurality of objects. For instance, referring to FIG. 5, in some embodiments, the graphical user interface allows for tracking the location of the container (e.g., block 520).

Furthermore, in some embodiments, the location associated with the corresponding object is visualized through a position in the order or organized objects, such as repositing a first object as an initial object in the order to be a final object in the order. However, the present disclosure is not limited thereto.

Referring to block 524, in some embodiments, the method 50 includes further displaying, through the graphical user interface of the display, an updated representation of the workflow. In some embodiments, the updated representation includes some or all of the plurality of objects displayed in a second sequence different from the first sequence.

In some embodiments, the method 50 further includes saving the updated representation of the workflow, such as to a memory of the computer system 100. For instance, in some embodiments, the end-user can save the representation or the updated representation or communicate the representation or the updated representation via a communication network 112. In some embodiments, the representation or the updated representation is stored in an isolated environment for that end-user so no other end-user can access another's workflows. However, the present disclosure is not limited thereto.

Accordingly, the method 50 allows an end-user to easily modify and create an updated representation of the workflow, such as via a simple click and drag input 110, the behavior of the one or more instruments 52 and/or the articulated handling robot 302 to produce a cell therapy.

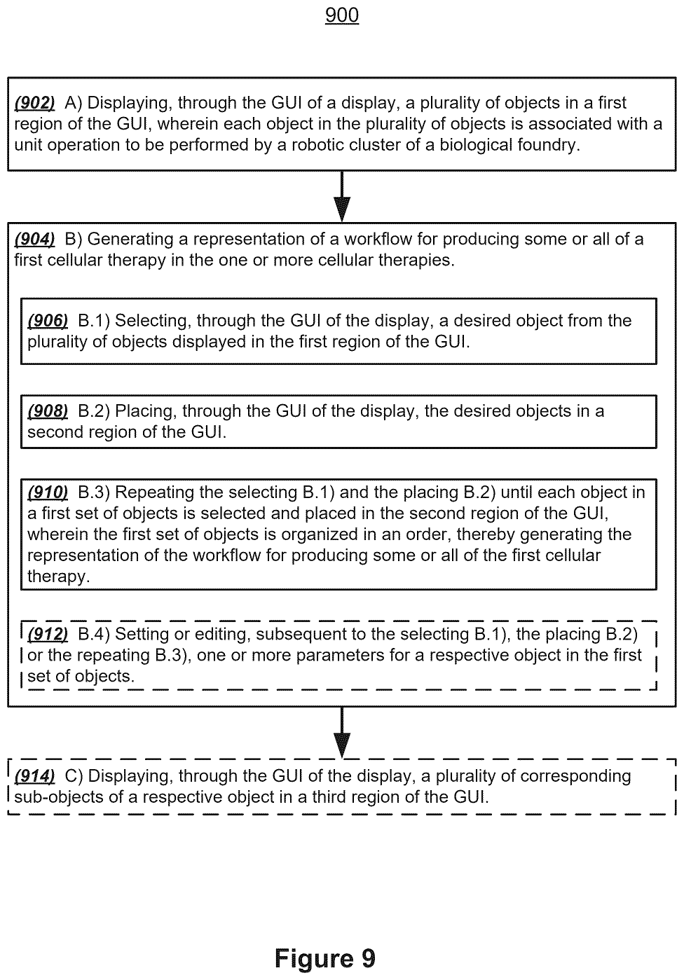

Referring to FIG. 9, there is depicted a flowchart illustrating a method, generally designated 900, in accordance with some exemplary embodiments of the present disclosure. In the flowchart, the preferred parts of the method are shown in solid line boxes, whereas additional, optional, or alternative parts of the method are shown in dashed line boxes. It should be noted that the processes disclosed herein and exemplified in the flowchart can be, but do not have to be, executed in full or in the order as they are presented.

The method 900 is configured for facilitating automated manufacture of one or more cellular therapies through a graphical user interface (GUI), such as the graphical user interface(s) illustrated in FIG. 2, FIG. 3, FIG. 4, and/or FIG. 5. In some embodiments, a cellular therapy in the one or more cellular therapies is an assembly of nucleic acid components. For instance, in some embodiments, the cellular therapy is a plasmid, and the nucleic acid components are predetermined promoters, repressors, stop codon, and exons. In some embodiments, the cellular therapy is a different predetermined nucleic acid with a different predetermined nucleic acid sequence. In some embodiments, the cellular therapy is a different predetermined ribonucleic acid (mRNA) with a different predetermined nucleic acid sequence. In some embodiments, the cellular therapy is a different predetermined deoxyribonucleic acid (DNA) with a different predetermined nucleic acid sequence. In some embodiments, the cellular therapy is a different predetermined polymer. In some embodiments, the cellular therapy is a different predetermined peptide. In some embodiments, the cellular therapy is a different predetermined protein. As such, in the context of biological foundries, the cellular therapy is one of the objectives of a research and development project that defines the desired biological trait to be achieved by a workflow. The cellular therapy can be either quantitative or qualitative. For example, in one embodiment, the cellular therapy can be a genetic configuration for a biosynthetic pathway that produces more compound of interest than a current level. In another embodiment, the cellular therapy is a genetic configuration for a microbial host that has a tolerance to an inhibitor over X mg/L. Additionally, in some embodiments, the cellular therapy is a polynucleotide or nucleic acid sequence.

In some embodiments, the method 900 and/or the GUI of the present disclosure enables a user to design a manufacturing process for a single lot from basic building blocks (e.g., objects). In some embodiments, through the GUI, the method 900 specifies operations for a robotic cluster of a biological foundry to perform in order to manufacture a therapy. In some embodiments, an optimization algorithm is employed to scale the process (e.g., a process represented by a workflow) to allow the robotic cluster to efficiently manufacture multiple lots in parallel. In some embodiments, a workflow is made up of a series of objects, where an object is the most basic building block and, in some cases, represents a series of actions the robotic cluster(s) will perform. In some embodiments, an object can be added to the workflow by click and drag from a bin or bank of objects. In some embodiments, one or each object is editable, which can affect the operations the robotic cluster(s) will perform. In some embodiments, an object can contain subprocesses made up of other objects. This enables organization of complex processes. In some embodiments, the method tracks the volume of a culture in the bioreactor(s) as reagents are added and removed. This is useful for understanding cell concentrations and/or avoid overflowing. In some embodiments, the method tracks the location of a cell culture. The method 900 and/or the GUI of the present disclosure allows a user to select, edit and/or organize desired objects, thereby allowing a user to easily “program” the behavior of the robotic cluster(s) to produce their own cell therapy product. In some embodiments, the user can save the representation of the workflow to a storage medium for that customer, such as the cloud that is stored in an isolated environment, so that no other customer can access another customer's processes.

Referring to block 902, in some embodiments, the method 900 includes A) a plurality of objects in a first region of the GUI, wherein each object in the plurality of objects is associated with a unit operation to be performed by a robotic cluster of a biological foundry. For instance, as a non-limiting examples, FIG. 2 illustrates a plurality of objects, such as the objects 210-1, 210-2, 210-3, . . . 210-j, that are displayed in a first region, such as the first region 201, of the GUI. The plurality of objects displayed in the first region of the GUI can include at least 2, at least 3, at least 4, at least 5, at least 6, at least 7, at least 8, at least 9, or at least 10 objects. In some embodiments, the plurality of objects displayed in the first region of the GUI includes at least 5, at least 10, at least 15, at least 20, at least 25, at least 30, at least 35, at least 40, at least 45, or at least 50 objects. While the objects in the figures appear as rectangular or substantially rectangular shapes, it should be noted that this is by way of illustration and the present disclosure is not limited thereto. An object can be of any suitable representation, including but not limited to one or more images, one or more symbols, one or more pictures, one or more tiles, one or more regular shapes, one or more irregular shapes, one or more graphical icons, or any combination thereof. An object can also include one or more indicia, such as one or more texts, one or more shades of a color, one or more hues of a color, or the combination thereof, that indicate the basic function(s) associated with the object. In some embodiments, the plurality of objects is displayed in the first region of the GUI in a row, an array, or the like. In some embodiments, the plurality of objects displayed in the first region of the GUI is referred herein as a bin or a bank of objects.

In some embodiments, each object in the plurality of objects is associated with a unit operation to be performed by one or more devices in a biological foundry. For instance, in some embodiments, the plurality of objects includes one or more enrich objects, one or more expand objects, one or more transfer objects, one or more harvest objects, one or more freeze objects, one or more formulate objects, one or more add reagent objects, one or more concentration-based sample objects, one or more fixed volume sample objects, or any combination thereof. In some embodiments, an expand object is associated with a unit operation configured to grow cells in a bioreactor (e.g., a first bioreactor). In some embodiments, a transfer object is associated with a unit operation configured to transfer a culture to a new bioreactor (e.g., from the first bioreactor to a second bioreactor). In some embodiments, a harvest object is associated with a unit operation configured to transfer expanded cells out of a bioreactor (e.g., the first or second bioreactor). In some embodiments, a freeze object is associated with a unit operation configured to freeze one or more final cell products. In some embodiments, a formulate object is associated with a unit operation configured to add a media (e.g., a cryopreservation media) and/or concentrate the culture.

In some embodiments, each respective object in the plurality of objects is associated with a corresponding module, or container, that accommodates a single instrument 52, which allows for the end-user to configure the workflow on an instrument, by instrument, basis. However, the present disclosure is not limited thereto. In some embodiments, each respective object in the plurality of objects is associated with at least two instruments 52. However, the present disclosure is not limited thereto.

For instance, in some embodiments, the respective object allows for the subject to modify and/or configuration operations and/or task performance using the instrument 52 by similar configuring one or more parameters associated with the respective object, such as a position of the respective object or a parameter of the respective object. However, the present disclosure is not limited thereto.

In some embodiments, a unit operation is to be performed by a robotic cluster of a biological foundry, such as the biological foundry 700 illustrated in FIG. 7. In some embodiments, the biological foundry includes at least one manufacturing cluster associated with a corresponding biological foundry operation, also referred to herein as a “robotic cluster,” which includes one or more instruments, such as one or more instruments 52-1, 52-2, etc., that perform the corresponding biological foundry operation. For instance, each robotic cluster of one or more instruments associated with the corresponding biological foundry operation includes at least one manufacturing instrument which performs some manufacturing step to produce a plurality of organic engineering targets at the biological foundry, and, optionally, a transport system, such as a convey system, that moves a portion of the manufacture of the plurality organic engineering targets, also referred to as a Work-In-Progress (WIP), between instruments in the one or more instruments of the biological foundry in a distributed and/or parallelized fashion. Each instrument is connected to a main control system (e.g., a controller and a communications interface) and to the other equipment via the controller at the biological foundry, and is given one or more execution instructions to perform work that products the plurality of organic engineering targets. In some embodiments, each instrument transmits out the data pertaining to the work a respective instrument performs for a corresponding workflow at the biological foundry. Each instrument is monitored locally at the biological foundry and by a remote device, such as a cloud server service. In some embodiments, a novel computer system architecture and method is provided to monitor and deploy the one or more instructions at the biological foundry in a scalable manner. This architecture of the present disclosure also ensures security and flexibility of the manufacturing system through the systems and methods described throughout the present disclosure. In some embodiments, there is one or more corresponding instruments 52, or action for each unit operation. In some embodiments, each instrument 52 is controlled by a standardized control module attached to the respective instrument 52. This control module receives a plurality of commands and transmits data about a status of executing an instant of a corresponding workflow conducted by the instrument 52. In some embodiments, a respective instrument 52 will perform different operations depending on the commands provided to the respective instrument 52.

Referring to blocks 604-610, in some embodiments, the method 900 includes B) generating a representation of a workflow for producing some or all of a first cellular therapy in the one or more cellular therapies. In some embodiments, the generating B) includes B.1) selecting, through the GUI of the display, a desired object from the plurality of objects displayed in the first region of the GUI, B.2) placing, through the GUI of the display, the desired objects in a second region of the GUI, and B.3) repeating the selecting B.1) and the placing B.2) until each object in a first set of objects is selected and placed in the second region of the GUI, wherein the first set of objects is organized in an order, thereby generating the representation of the workflow for producing some or all of the first cellular therapy.

For instance, as a non-limiting example, FIG. 2 illustrates selecting the object 210-1 and placing it in a second region, such as a second region 202, of the GUI. As another non-limiting example, FIG. 3 illustrates selecting the object 210-5 and placing it in the second region of the GUI after the objects 210-1 and 210-2 have been selected and placed in the second region of the GUI. As a further non-limiting example, FIG. 4 illustrates a first set of objects has been selected and placed in the second region of the GUI. In this non-limiting example, the first set of objects includes the objects 210-1, 210-4, 210-5 etc. and is organized in an order, thereby generating the representation of the workflow for producing some or all of a first cellular therapy. The organization of the first set of objects in an order can be conducted while performing the selecting, placing and/or repeating steps, or after the first set of objects has been selected and placed in the second rejoin of the GUI.

The appearance (e.g., size, shape, color, etc.) of an object in the second region of the GUI can be the same as or different from that in the first region of the GUI. As a non-limiting example, FIG. 4 illustrates that the appearance of an object changes (e.g., in size, shape, indicia, etc.) when placed in the second region of the GUI.

The selecting of a desired object and the placing of the desired object in the second region of the GUI can be made, for instance, through the one or more input device(s) 110 of the computer system 100, or by other mechanisms such as touch or audio commands. For instance, in some embodiments, the selecting of a desired object and the placing of the desired object in the second region of the GUI are conducted by clicking the desired object in the plurality of objects displayed in the first region of the GUI and dragging the desired object to the second region of the GUI. As such, the present disclosure provides a useful tool that allows a user to easily “program” the behavior of the robotic cluster to produce their own cell therapy product.

Depending on the cellular therapy to be produced, the number and/or order of objects to be selected and placed in the second region of the GUI can vary. For instance, in an exemplary embodiment, the first set of objects consists of a single object selected from the plurality of objects. In another exemplary embodiment, the first set of objects consists of each and every object in the plurality of objects. In some embodiments, the first set of objects includes two or more objects selected from the plurality of objects.

In some embodiments, the workflow is configured to be performed independent of any other workflows. In some embodiments, the workflow is configured to be performed as part of a series workflow. In some embodiments, the workflow is configured to be performed as part of a parallel workflow, e.g., concurrently with a different workflow or a portion of the different workflow. In some such embodiments, the method 900 includes an optimization algorithm that scales the workflow to allow the robotic cluster(s) to efficiently manufacture multiple cellular therapies in parallel.

In some embodiments, the plurality of objects includes a first object (e.g., the object 210-1) that is associated with a first instruction to start the workflow, a second object (e.g., the object 210-2) that is associated with a second instruction to end the workflow, or both of the first object and the second object. In some such embodiments, the first set of objects includes the first object, the second object, or both of the first object and the second object.

Referring to block 612, in some embodiments, the generating of the representation of the workflow for producing some or all of the first cellular therapy further includes B.4) setting or editing one or more parameters for a respective object in the first set of objects. The setting or editing of the one or more parameters for a respective object can be conducted after selecting a desired object, placing the desired object in the second region of the GUI, or after repeating the selecting step and the placing step one or more times. The setting or editing the one or more parameters for a respective object allows for affecting the operation(s) the robotic cluster (e.g., one or more instruments 52 and/or articulated handling robot 302) will perform when manufacturing the first cell therapy.

Different objects can have the same or different type(s) of parameters. Different objects can have the same or different number(s) of parameters. For instance, in an exemplary embodiment, an object consists of a single parameter. In some embodiments, an object includes a plurality of parameters (e.g., 2, 3, 4, 5 or more than 5 parameters). As a non-limiting example, FIG. 2 illustrates that the object 210-1 includes a plurality of parameters, such as a parameter 211 for providing a description, a parameter 212 for specifying a volume, a parameter 213 for specifying a culture location, and a parameter 214 for specifying a device or module location. However, the present disclosure is not limited thereto.

Referring to block 614, in some embodiments, the method 900 includes C) Displaying, through the GUI of the display, a plurality of corresponding sub-objects of a respective object in a third region of the GUI. For instance, in some embodiments, an object includes a plurality of corresponding sub-objects. In some embodiments, a corresponding sub-object is an object selected from the plurality of objects. This allows for grouping or organizing complex processes through hierarchies of objects. As a non-limiting example, FIG. 5 illustrates an “Expand” object (e.g., the object 210-4) in the plurality of objects includes one or more subprocesses made of an “Input Bioreactor” object (e.g., the object 210-4-1), an “Add Reagent” object (e.g., the object 210-4-2), a “Fixed Volume Sample” object (e.g., the object 210-4-3), and an “Output Bioreactor” object (e.g., the object 210-4-4) that are displayed in a third region 203 of the GUI. In some embodiments, the displaying of the plurality of corresponding sub-objects of a respective object in the third region of the GUI is in response to a selection of the respective object presented in the second region of the GUI, for instance, by clicking the respective object presented in the second region of the GUI.

In some embodiments, the method 900 and/or the GUI allows for tracking the location and/or volume of a fluid (e.g., culture or the like). For instance, as a non-limiting example, FIG. 5 illustrates tracking a culture volume 222 in two containers 223, e.g., the containers labeled “GREX-ETM” and “GREX-ICM,” as reagents are added and/or removed from the container(s). This is useful to understand fluid concentrations and/or avoid overgrowth when manufacturing the first cell therapy.

In some embodiments, the method 900 further includes saving the representation of the workflow, for instance, to a memory of the computer system 100. In some embodiments, the end-user can save the representation or communicate the representation via a communication network 112. In some embodiments, the representation stored in an isolated environment for that end-user so no other end-user can access another's workflows. However, the present disclosure is not limited thereto. Accordingly, the method 900 allows an end-user to easily access, modify and/or create an updated representation of the workflow, such as via a simple click and drag input 110, the behavior of the one or more instruments 52 and/or the articulated handling robot 302 to produce a cell therapy.

The method 900 and/or the GUI of the present disclosure can include additional, optional and/or alternative features. For instance, the GUI can include one or more input controls configured to get information from the user, one or more navigational components configured to control movement from one GUI to another, one or more informational components configured to deliver information about task status or system information, one or more elements such as buttons, dropdown lists, and windows, or any combination thereof. As a non-limiting example, FIG. 2 illustrates the GUI includes a logo/name feature 204, a process name feature 205, a batch record feature 206, and a confirm feature 215.

In some embodiments, the systems and methods of the present disclosure provide a unique, modular approach to cell therapy automatic that reduces manufacturing bottle necks and minimizes human contamination, such as by allowing for a subject to generating a representation of a workflow for producing some or all of a first therapy using a computer system.

### Illustration of Subject Technology as Clauses

Various example of aspects of the disclosure are described as numbered clauses (e.g., 1, 2, 3, etc.) for convenience. These are provided as examples, and do not limit the subject technology.

Clause 1: A method for facilitating automated manufacture of one or more cellular therapies through a graphical user interface (GUI), the method comprising: A) displaying, through the GUI of a display, a plurality of objects in a first region of the GUI, wherein each object in the plurality of objects is associated with a unit operation to be performed by a robotic cluster of a biological foundry; and B) generating a representation of a workflow for producing some or all of a first cellular therapy in the one or more cellular therapies, wherein the generating comprises: B.1) selecting, through the GUI of the display, a desired object from the plurality of objects displayed in the first region of the GUI; B.2) placing, through the GUI of the display, the desired objects in a second region of the GUI; and B.3) repeating the selecting B.1) and the placing B.2) until a first set of objects is selected and placed in the second region of the GUI, wherein the first set of objects is organized in an order, thereby generating the representation of the workflow for producing some or all of the first cellular therapy.

Clause 2: The method of clause 1, wherein the plurality of objects comprises: a first object that is associated with a first instruction to start the workflow; a second object that is associated with a second instruction to end the workflow; or both of the first object and the second object.

Clause 3: The method of clause 2, wherein the first set of objects comprises the first object, the second object, or both of the first object and the second object.

Clause 4: The method of any one of clauses 1-3, wherein the first set of objects comprises two or more objects selected from the plurality of objects.

Clause 5: The method of any one of clauses 1-4, wherein the first set of objects consists of each object in the plurality of objects.

Clause 6: The method of any one of clauses 1-5, wherein the plurality of objects comprises one or more enrich objects, one or more expand objects, one or more transfer objects, one or more harvest objects, one or more freeze objects, one or more formulate objects, one or more add reagent objects, one or more concentration-based sample objects, one or more fixed volume sample objects, or any combination thereof.

Clause 7: The method of any one of clauses 1-6, wherein the selecting B.1) and the placing B.2) are conducted by clicking the desired object in the plurality of objects displayed in the first region of the GUI and dragging the desired object to the second region of the GUI.

Clause 8: The method of any one of clauses 1-7, wherein the generating further comprises: B.4) setting or editing, subsequent to the selecting, the placing or the repeating, one or more parameters for a respective object in the first set of objects.

Clause 9: The method of any one of clauses 1-8, wherein a respective object in the plurality of objects comprises a plurality of corresponding sub-objects.

Clause 10: The method of clause 9, further comprising: C) displaying, through the GUI of the display, the plurality of corresponding sub-objects of the respective object in a third region of the GUI.

Clause 11: A method for facilitating automated manufacture of one or more cellular therapies through a graphical user interface, the method comprising: displaying, through the graphical user interface of a display, a representation of a workflow that is configured to produce some or all of a first cellular therapy in the one or more cellular therapies, wherein the representation comprises a plurality of objects displayed in a first sequence; receiving, through the graphical user interface of the display, a modification to the representation of the workflow; and further displaying, through the graphical user interface of the display, an updated representation of the workflow, wherein the updated representation comprises some or all of the plurality of objects displayed in a second sequence different from the first sequence.

Clause 12: The method of clause 11, wherein the representation of the workflow defines an order of operations for a remote device to perform.

Clause 13: The method of clause 12, wherein the order of operations comprises a sort operation, an edit operation, a culture operation, a quality control operation, a formulate operation, or a combination thereof.

Clause 14: The method of any one of clauses 11-13, wherein the order of operations is spatiotemporally organized.

Clause 15: The method of any one of clauses 11-14, wherein the workflow is configured to be performed concurrently with a different workflow.

Clause 16: The method of any one of clauses 11-15, wherein the plurality of objects comprises one or more tiles, one or more squares, one or more rectangles, one or more closed-form polygons, one or more circles, or more ellipses, or a combination thereof.

Clause 17: The method of any one of clauses 11-16, wherein each object in the plurality of objects is associated with a unit operation performed by the remote device.

Clause 18: The method of any one of clauses 11-17, wherein the receiving the modification further comprises determining if the modification satisfies one or more heuristic constraints.

Clause 19: The method of any one of clauses 11-18, wherein the modification comprises a reagent volume associated with a corresponding object in the plurality of objects.

Clause 20: The method of any one of clauses 11-19, wherein the modification comprises a location associated with a corresponding object in the plurality of objects.

Clause 21: A computer system comprising a display, one or more processors, and memory, the memory storing instructions for executing the method of according to any one of clauses 1-20.

Clause 22: A computer readable storage medium storing one or more programs, the one or more programs comprising instructions, which when executed by an electronic device with one or more processors and a memory cause the electronic device to execute the method of according to any one of clauses 1-20.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that includes a computer program mechanism embedded in a non-transitory computer-readable storage medium. For instance, the computer program product could contain instructions for operating the user interfaces disclosed herein and described with respect to the Figures. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

What is claimed is:

1. A method for facilitating automated manufacture of one or more cellular therapies through a graphical user interface (GUI), the method comprising:
A) displaying, through the GUI of a display, a plurality of objects in a first region of the GUI, wherein each object in the plurality of objects is associated with a unit operation to be performed by a robotic cluster of a biological foundry; and
B) generating a representation of a workflow for producing some or all of a first cellular therapy in the one or more cellular therapies, wherein the generating comprises:
B.1) selecting, through the GUI of the display, a desired object from the plurality of objects displayed in the first region of the GUI;
B.2) placing, through the GUI of the display, the desired objects in a second region of the GUI; and
B.3) repeating the selecting B.1) and the placing B.2) until each object in a first set of objects is selected and placed in the second region of the GUI, wherein the first set of objects is organized in an order, thereby generating the representation of the workflow for producing some or all of the first cellular therapy.

2. The method of claim 1, wherein the plurality of objects comprises:
a first object that is associated with a first instruction to start the workflow;
a second object that is associated with a second instruction to end the workflow; or
both of the first object and the second object.

3. The method of claim 2, wherein the first set of objects comprises the first object, the second object, or both of the first object and the second object.

4. The method of claim 1, wherein the first set of objects comprises two or more objects selected from the plurality of objects.

5. The method of claim 1, wherein the first set of objects consists of each object in the plurality of objects.

6. The method of claim 1, wherein the plurality of objects comprises one or more enrich objects, one or more expand objects, one or more transfer objects, one or more harvest objects, one or more freeze objects, one or more formulate objects, one or more add reagent objects, one or more concentration-based sample objects, one or more fixed volume sample objects, or any combination thereof.

7. The method of claim 1, wherein the selecting B.1) and the placing B.2) are conducted by clicking the desired object in the plurality of objects displayed in the first region of the GUI and dragging the desired object to the second region of the GUI.

8. The method of claim 1, wherein the generating further comprises:
B.4) setting or editing, subsequent to the selecting, the placing or the repeating, one or more parameters for a respective object in the first set of objects.

9. The method of claim 1, wherein a respective object in the plurality of objects comprises a plurality of corresponding sub-objects.

10. The method of claim 9, further comprising:
C) displaying, through the GUI of the display, the plurality of corresponding sub-objects of the respective object in a third region of the GUI.

11. A method for facilitating automated manufacture of one or more cellular therapies through a graphical user interface, the method comprising:
displaying, through the graphical user interface of a display, a representation of a workflow that is configured to produce some or all of a first cellular therapy in the one or more cellular therapies, wherein the representation comprises a plurality of objects displayed in a first sequence;
receiving, through the graphical user interface of the display, a modification to the representation of the workflow; and
further displaying, through the graphical user interface of the display, an updated representation of the workflow, wherein the updated representation comprises some or all of the plurality of objects displayed in a second sequence different from the first sequence.

12. The method of claim 11, wherein the representation of the workflow defines an order of operations for a remote device to perform.

13. The method of claim 12, wherein the order of operations comprises a sort operation, an edit operation, a culture operation, a quality control operation, a formulate operation, or a combination thereof.

14. The method of claim 11, wherein the order of operations is spatiotemporally organized.

15. The method of claim 11, wherein the workflow is configured to be performed concurrently with a different workflow.

16. The method of claim 11, wherein the plurality of objects comprises one or more tiles, one or more squares, one or more rectangles, one or more closed-form polygons, one or more circles, or more ellipses, or a combination thereof.

17. The method of claim 11, wherein each object in the plurality of objects is associated with a unit operation performed by the remote device.

18. The method of claim 11, wherein the receiving the modification further comprises determining if the modification satisfies one or more heuristic constraints.

19. The method of claim 11, wherein the modification comprises a reagent volume associated with a corresponding object in the plurality of objects.

20. The method of claim 11, wherein the modification comprises a location associated with a corresponding object in the plurality of objects.
